(19) United States
(12) Patent Application Publication
(10) Pub. No.: US 2011/0245548 A1
(43) Pub. Date: Oct. 6, 2011

(54) CATALYST LIFE IMPROVEMENT IN THE VAPOR PHASE FLUORINATION OF CHLOROCARBONS

(75) Inventors: DANIEL C. MERKEL, Orchard Park, NY (US); KONSTANTIN A. POKROVSKI, Orchard Park, NY (US); HSUEH S. TUNG, Getzville, NY (US)

(73) Assignee: HONEYWELL INTERNATIONAL INC., Morristown, NJ (US)

(21) Appl. No.: 13/042,589

(22) Filed: Mar. 8, 2011

Related U.S. Application Data
(60) Provisional application No. 61/319,640, filed on Mar. 31, 2010.

(51) Int. Cl. C07C 17/25; C07C 17/35

(52) U.S. Cl. 570/156; 570/160

(57) ABSTRACT

The present invention achieves a catalyst life improvement for the catalyzed vapor phase fluorination of a chlorocarbon in the presence of one catalyst and an oxygen feed. Specifically, in one non-limiting embodiment, the instant invention provides the conversion of 1,1,2,3-tetrachloropropene and/or 1,1,1,2,3-pentachloropropane to 2-chloro-3,3,3-trifluoropropene by introduction of a catalyst and oxygen co-feed to the fluorination reactor.

### CROSS REFERENCE TO RELATED APPLICATIONS

This application is related to and claims the priority benefit of U.S. provisional application number 61/319,640 filed Mar. 31, 2010, the contents of which are incorporated herein by reference.

### FIELD OF THE INVENTION

The present invention relates to a method for improving the life of a catalyst during vapor phase fluorination of chlorocarbons such as, but not limited to, fluorination of 1,1,2,3-tetrachloropropene (HCO-1230xa) and/or 1,1,1,2,3-pentachloropropane (HCC-240db) to 2-chloro-3,3,3,-trifluoropropene (HCFO-1233xf).

### BACKGROUND OF THE INVENTION

Fluorocarbon based fluids have found widespread use in industry in a number of applications, including as refrigerants, aerosol propellants, blowing agents, heat transfer media, and gaseous dielectrics. Because of the suspected environmental problems associated with the use of some of these fluids, including the relatively high global warming potentials associated therewith, it is desirable to use fluids having the lowest possible greenhouse warming potential in addition to zero ozone depletion potential. Thus there is considerable interest in developing environmentally friendlier materials for the applications mentioned above.

Tetrafluoropropenes, having essentially zero ozone depletion and low global warming potential, have been identified as potentially filling this need. However, the toxicity, boiling point, and other physical properties in this class of chemicals vary greatly, even between different isomers of a compound. One tetrafluoropropene having valuable properties is 2,3,3,3-tetrafluoropropene (HFO-1234yf). This compound has been found to be an effective refrigerant, heat transfer medium, propellant, foaming agent, blowing agent, gaseous dielectric, sterilant carrier, polymerization medium, particulate removal fluid, carrier fluid, buffing abrasive agent, displacement drying agent and power cycle working fluid.

There are a multitude of known processes for the production of tetrafluoropropenes. One process, for example, involves the use of tetrachloropropenes as a reactant in the conversion to the desired C3 compound (US 2007/0197842 A1). Other methods include those disclosed in U.S. Pat. No. 4,900,874 (describing a method of making fluorine containing olefins by contacting hydrogen gas with fluorinated alcohols); U.S. Pat. No. 2,931,840 (describing a method of making fluorine containing olefins by pyrolysis of methyl chloride and tetrafluoroethylene or chlorodifluoromethane); U.S. Pat. No. 5,162,594 (disclosing a process wherein tetrafluoroethylene is reacted with another fluorinated ethylene in the liquid phase to produce a polyfluoroolefin product); and Banks, et al., Journal of Fluorine Chemistry, Vol. 82, Iss. 2, p. 171-174 (1997) (disclosing the preparation of HFO-1234yf from trifluoroacetylacetone and sulfur tetrafluoride). A manufacturing process for producing 2,3,3,3-tetrafluoropropene (1234yf), in particular, requires the fluorination of either 1,1,2,3-tetrachloropropene or 1,1,1,2,3-pentachloropropane (HCC-240db) with Hydrogen Fluoride to form 2-chloro-3,3,3-trifluoropropene (HCFO-1233xf), which is a well known intermediate in the production of 2,3,3,3-tetrafluoropropene (HFO-1234yf) and is described in U.S. Applications 20070007488, 20070197842, and 20090240090, the contents of which are incorporated herein by reference. This reaction takes place in the vapor phase using a fluorination catalyst of partially fluorinated Cr₂O₃. While initial reaction studies discovered that the catalyst life was very short in comparison to other fluorination reactions that incorporate the same catalyst, U.S. Patent Application No. 20090030244, the contents of which are incorporated herein by reference, illustrated that the addition of a stabilizer to the reaction extends the catalyst life by a minimum of 8 fold.

Even in view of the foregoing references, there continues to be a need for improving the cost efficiency of such reactions. For example, many of the foregoing references disclose processes that involve separate steps as well as disparate reaction conditions, reagents, and catalysts. The efficiency of such multi-step processes is limited by the efficiency of each individual step. One inefficient step, such as the shortened life of the requisite catalyst, may make the entire process more resource intensive, less effective at converting intermediates to the desired fluorocarbon products and less productive, suffering yield losses due to increased impurity formation. Against this backdrop, there is a continuing need for less resource-intensive processes that produce increased conversion of intermediates to the end product halogenated olefin over a longer period due, in particular, to substantially increased catalyst life. To this end, there is a continuing need for methods of efficiently preparing intermediates of certain hydrohalocarbons, particularly compounds which are in part useful as intermediates in the preparation of tetrafluoropropenes, such as 2,3,3,3-tetrafluoropropene (HFO-1234yf). The instant invention and the embodiments presented herein address such a need.

### SUMMARY OF THE INVENTION

The present invention relates to a method of preparing fluorinated organic compounds comprising contacting at least one chlorocarbon, such as tetrachloropropene or pentachloropropane, with a halogenating agent in the presence of at least one catalyst and an oxygen containing feed under conditions effective to produce a C3 haloolefin.

In one embodiment, the chlorocarbon is 1,1,2,3-tetrachloropropene, 1,1,1,2,3-pentachloropropane, or combinations thereof and the final C3 haloolefin is 2-chloro-3,3,3,-trifluoropropene. The halogenating agent may include any fluorinating agent, such as but not limited to hydrogen fluoride. The mole ratio of the halogenating agent to the chlorocarbon is greater than or equal to 3:1, where in certain embodiments it is between 5:1 and 20:1. The mole ratio of the oxygen feed to the chlorocarbon may be provided by a feed stream to be less than or equal to 0.1:1, where in certain embodiments it is between 0.07:1 and 0.005:1 or between 0.01:1 and 0.05:1.

In certain embodiments of these methods, the source of oxygen may be selected from the group consisting of oxygen gas, dry air, or oxygen gas diluted with an inert gas such as, but not limited to, nitrogen, argon, or helium.

The catalysts used in the instant reaction may be one or a combination of fluorination catalysts. Suitable catalysts include, but are not limited to, chromium, aluminum, cobalt, manganese, nickel and iron oxides, hydroxides, halides, oxyhalides, inorganic salts thereof and their mixtures. Examples of such catalysts include, but are not limited to, Cr₂O₃, FeCl₃/C, Cr₂O₃, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, NiCl₂/AlF₃, SnCl₄/C, TaCl₅/C, SbCl₃/C, AlCl₃/C, AlF₃/C and combinations thereof. In certain embodiments, the fluorination catalyst is Cr₂O₃. All of the listed catalysts may be partially or totally fluorinated by anhydrous HF.

In one preferred method, the catalyst comprises one or more chromium (III) oxides. Preferably, the catalyst comprises amorphous chromium oxide. In most preferred embodiment, the catalyst is at least partially, if not fully, fluorinated.

In further embodiments, at least a portion of the step of contacting the chlorocarbon with the halogenating agent is conducted in the gas phase. In further embodiments, the step of contacting the chlorocarbon with the halogenating agent is conducted at a temperature of from about 150° C. to about 450° C. and/or at a pressure of from about 0 to about 200 psig.

The instant invention is advantageous because the presence of the oxygen feed surprisingly extends the life of the catalyst for a period of time greater than when the oxygen feed is not present. Thus, the instant invention allows for greater conversion to the final C3 haloolefin. In even further embodiments, the catalyst in the presence of the oxygen feed is substantially operable at least about two fold longer than said catalyst wherein said oxygen feed is not present.

Additional embodiments and advantages of the instant invention will be readily apparent to one of ordinary skill in the art based on the disclosure provided herein.

### BRIEF DESCRIPTION OF THE FIGURES

FIG. 1 illustrates the temperature reaction of HCO-1230xa with hydrogen fluoride in the presence of a Cr₂O₃ catalyst and oxygen co-feed.

FIG. 2 illustrates the temperature reaction of HCO-1230xa with hydrogen fluoride in the presence of a Cr₂O₃ catalyst without the oxygen co-feed.

### DETAILED DESCRIPTION OF THE INVENTION

The instant invention relates, at least in part, to the discovery of the correlation between the rate of catalyst deactivation during the reaction of one or more chlorocarbons with a halogenating agent and the rate of the temperature change inside the catalyst bed. More specifically, an active catalyst exhibits a large exotherm relative to the external reactor heater. As the catalyst deactivates, the exotherm diminishes and the temperature inside the deactivated catalyst bed approaches that of the external heater. It has been surprisingly found that the life of the catalyst during fluorination can be increased by at least two fold if an oxygen co-feed is introduced into the fluorination reactor together with the feed(s) of the raw materials. Slower catalyst deactivation with oxygen co-feed minimizes the loss in production time due to the need to regenerate the catalyst off-line.

In one embodiment, the methods of the present invention comprise reacting one chlorocarbon or mixed chlorocarbon feed material with a fluorinating agent to produce a fluorinated haloolefin, preferably a C3 fluorinated haloolefin. While not limited thereto, in one embodiment, the chlorocarbons may be a tetrachloropropene and/or a pentachloropropane compound, and the C3 fluorinated haloolefin is a trifluoropropene compound. In an even further non-limiting embodiment the chlorocarbons are 1,1,2,3-tetrachloropropene (HCO-1230xa) and/or 1,1,1,2,3-pentachloropropane (HCC-240db) and the C3 fluorinated haloolefin is 2-chloro-3,3,3-trifluoropropene (HCFO-1233xf). The reaction steps for producing HFC-1233xf may be described, by way of illustration but not necessarily by way of limitation, by the following two reaction equations:

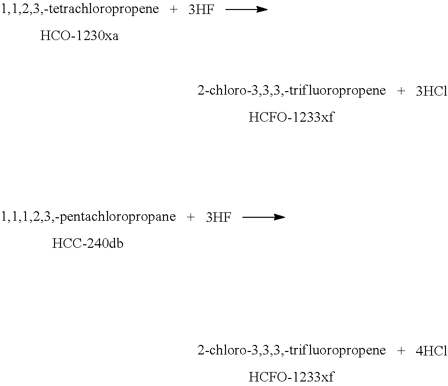

Such reactions exemplify a continuous or batch method for producing 2-chloro-3,3,3,-trifluoropropene (HCFO-1233xf) by vapor phase fluorination of one chlorocarbon or mixed chlorocarbon feed material of 1,1,1,2,3-pentachloropropane (HCC-240db) and/or 1,1,2,3,-tetrachloropropene (HCO-1230xa) with hydrogen fluoride to produce a stream comprising hydrogen fluoride, 2-chloro-3,3,3,-trifluoropropene and hydrogen chloride.

The instant fluorination reactions may be conducted in any reactor suitable for a vapor or liquid phase fluorination reaction. In certain embodiments, the reactor is constructed from materials which are resistant to the corrosive effects of hydrogen fluoride and a catalyst such as Hastalloy, Inconel, Monel and vessels lined with fluoropolymers, which are generally known in the art. In case of a vapor phase process, the reactor is filled with a vapor phase fluorination catalyst, which may include any fluorination catalysts known in the art. Suitable catalysts include, but are not limited to, chromium, aluminum, cobalt, manganese, nickel and iron oxides, hydroxides, halides, oxyhalides, inorganic salts thereof and their mixtures. Combinations of catalysts suitable for the present invention nonexclusively include Cr₂O₃ Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃ NiCl₂/AlF₃ and mixtures thereof. Additional fluorination catalysts that can be used include FeCl₃/C, SnCl₄/C, TaCl₅/C, SbCl₃/C, AlCl₃/C, and AlF₃/C. Support for these metal halides include alumina or fluorinated alumina bases or any otherwise known catalyst support known in the art. All of the listed catalysts may be partially or totally fluorinated by anhydrous HF prior to initiating the reaction.

While not limited thereto, chromium (III) oxides such as crystalline chromium oxide or amorphous chromium oxide are preferred catalysts with amorphous chromium oxide being most preferred. Amorphous chromium oxide (Cr₂O₃) is a commercially available material which may be purchased in a variety of particle sizes. Fluorination catalysts having a purity of at least 98% are preferred though also not limiting. The fluorination catalyst may be present in an excess but in at least an amount sufficient to drive the reaction. The catalysts can be supported or in bulk. The fluorination catalyst may be present in an excess but in at least an amount sufficient to drive the reaction.

Preferably the reactor is constructed from materials that are resistant to the corrosive effects of the HF and catalyst, such as Hastelloy-C, Inconel, Monel, Incolloy. Such vapor phase fluorination reactors are well known in the art.

In one non-limiting embodiment, a reactor may be loaded with a sufficient amount of desired vapor phase fluorination catalyst, wherein a sufficient amount is any amount necessary to drive the reaction. The reactor is then pre-heated to a temperature between about 30° C. to about 300° C., and in certain embodiments the reactor is pre-heated to about 225° C. The pressure of the reactor is also adjusted to be between about 0.0 psig to about 125 psig. In certain embodiments the pressure is about 2 psig. Optionally, an inert gas purge, such a nitrogen gas, may be provided over the catalyst after the reactor temperature has been increased but before the reactants are introduced.

The chlorocarbon(s), halogenating agents, and oxygen feed are then simultaneously pre-vaporized or preheated to a temperature of from about 30° C. to about 300° C. and are then fed to the reactor. Optionally, oxygen co-feed is introduced after chlorocarbon and fluorinating agent feeds are vaporized but before the fluorination reactor. During the vapor phase fluorination reaction, the reactants are reacted in a vapor phase in the presence of the fluorination catalyst and oxygen. The reactant vapor is allowed to contact the fluorination catalyst from about 1 to 120 seconds or more preferably from about 1 to 20 seconds. The instant invention, however, is not limited to such a contact time any may include any time required for the gaseous reactants to pass through the catalyst bed assuming that the catalyst bed is 100% void. The reactor effluent consisting of 1233xf, partially fluorinated intermediates and by-products, overfluorinated by-products, HF, and HCl exit the reactor and become available for recovery or further processing. Recovery and recycle of intermediates, e.g. HCFO-1232xf, 1231xf, and unreacted reactants may be accomplished using means known in the art.

The process or steps of contacting the reactants with the catalyst are not necessarily limited to the foregoing and the reaction steps may be provided in any order with any convenient temperature and pressure. In an alternative non-limiting embodiment, for example, oxygen co-feed can be introduced to the feed stream after the other reactants are pre-vaporized but before or simultaneous with the vaporized reactants being provided to the reactor. In an even further alternative, the reactants, with or without the presence of oxygen, are pre-vaporized in the reactor. Accordingly, modification of the order of performing the steps provided above are contemplated in the instant invention to achieve or otherwise optimize reaction conditions.

The reactant feeds may be adjusted to achieve the desired mole ratio by regulating flow rates into the reactor. In one embodiment, the mole ratio of halogenating agent (e.g. HF) to chlorocarbon (e.g. HCO-1230xa and/or HCC-240db) is ≧3:1. In alternative non-limiting embodiments, the mole ratio of halogenating agent (e.g. HF) to chlorocarbon (e.g. HCO-1230xa and/or HCC-240db) is between 3:1 and 20:1, between 4:1 and 12:1, or between 5:1 and 10:1.

The oxygen feed may similarly be adjusted to the desired mole ratio by adjusting flow rates into the reactor. In one non-limiting embodiment, the air co-feed is introduced at the rate that results in a O₂ to chlorocarbon (e.g. HCO-1230xa and/or HCC-240db) ratio of about 0.032:1. These flow rates may be adjusted, however, to achieve alternative mole ratios of oxygen to chlorocarbons that are preferably, though not limited to, ≦0.1:1. In alternative non-limiting embodiments, the mole ratio of oxygen to chlorocarbon (e.g. HCO-1230xa and/or HCC-240db) is between 0.07:1 and 0.005:1, or between 0.01:1 and 0.05:1.

Although non-limiting to the invention, the vapor phase fluorination reaction is conducted at a temperature ranging from about 150° C. to about 450° C. In alternative non-limiting embodiments, the temperature range is between 175° C. to about 425° C. between 200° C. to about 400° C., between 225° C. to about 390° C., or between 250° C. to about 380° C.

While the reactor pressure is not critical and can be superatmospheric, atmospheric or under vacuum, in one embodiment the reaction pressure is between about 0.0 psig to about 200 psig. In alternative non-limiting embodiments, the pressure range is between about 0 to 150 psig, or between about 2 to about 125 psig.

In certain embodiments, the present step of fluorinating a chlorocarbon to produce a C3 haloolefin comprises contacting the chlorocarbon with a fluorinating agent, preferably under conditions effective to provide a conversion rate of at least about 50%, more preferably at least about 55%, and even more preferably at least about 70%. In further embodiments, the conversion is at least about 90% or about 100%. In embodiments in which the chlorocarbon comprises HCO-1230xa or HCC-240db the selectivity to HCFO-1233xf is at least about 5%, at least about 20%, at least about 50%, or at least about 99%.

As noted above, previous methods of preparing fluorinated organic compounds in the presence of such catalysts required reaction shut down for the regeneration of the catalyst. The instant invention relates to the surprising discovery that oxygen co-feed may be provided to the reactor simultaneously with the reactants. This results in extending the life of the catalyst by a minimum of two fold, as compared to the catalyst life in the absence of the oxygen co-feed. Such an application is advantageous because it reduces the resource intensity required for conversion, thus, decreases reaction costs and costs associated with productivity.

The following non-limiting examples serve to illustrate the invention

### EXAMPLE 1

This example illustrates the continuous vapor phase fluorination reaction of 1,1,2,3-tetrachloropropane (HCO-1230xa)+3HF→2-chloro-3,3,3-trifluoropropene (1233xf)+4HC1 in the presence of oxygen co-feed. The fluorination catalyst for the experiment is fluorinated Cr₂O₃.

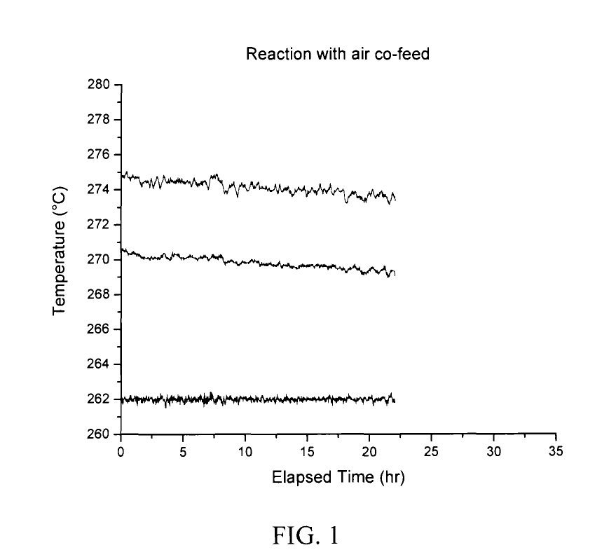

A continuous vapor phase fluorination reaction system consisting of air, N2, HF, and organic feed systems, feed vaporizer, superheater. 2″ ID monel reactor, acid scrubber, drier, and product collection system is used to study the reaction. The reactor is loaded with 2135 grams of pretreated Cr₂O₃ catalyst which equates to about 1.44 liters of catalyst (the total height of the catalyst bed is about 28 inches). A multipoint thermocouple is installed in the middle of the reactor. The reactor is then heated to a reaction temperature of about 225° C. with a N₂ purge going over the catalyst after the reactor has been installed in a constant temperature sand bath. The reactor is at about 2 psig of pressure. HF feed is introduced to the reactor (via the vaporizer and superheater) as a co-feed with the N₂ for 15 minutes when the N₂ flow is stopped. The HF flow rate is adjusted to 1.0 lb/hr and then 1,1,2,3-tetrachloropropene (HCO-1230xa) feed is started to the reactor (via the vaporizer and superheater) at 1.25 lb/hr. Then air co-feed is introduced (air flow is added before the vaporizer) at the rate of about 150 cm³/min resulting in a o₂ to HCO-1230xa ratio of about 0.032:1. The feed rate of HCO-1230xa is kept steady at about 1.25 lb/hr and HF feed is kept steady at 1.0 lb/hr for about a 7.2 to 1 mole ratio of HF to 1230xa. Once the reaction is started, the catalyst bed temperature is adjusted to about 270-280° C. The complete conversion of HCO-1230xa is observed throughout the experiment. During the experiment the catalyst bed temperature is higher than that of external reactor heater (sand bath, bottom line of FIG. 1) due to the exothermic character of the HCO-1230xa fluorination reaction. Also, since excess catalyst is used, a temperature gradient is observed throughout the catalyst bed. Initially, the highest temperature (hot-spot) is observed at the inlet of the reactor. The hot-spot position slowly moves through the catalyst bed as the continuous reaction progresses indicating at least a partial deactivation of the catalyst at the inlet of the reactor. After the reaction hot-spot moves to the middle of the reactor (total length of catalyst bed was about 28 inches) two points (11 and 14 inches from the reactor inlet) inside catalyst bed are selected to monitor the rate of catalyst deactivation. The temperatures at these two positions inside the catalyst bed are monitored for over 20 hours. It is calculated that the temperature at 11 inches (middle line of FIG. 1) was decreasing linearly at the rate of 0.04978° C./hr and the temperature at 14 inches (top line of FIG. 1) was decreasing linearly at the rate of 0.05053° C./hr (FIG. 1).

EXAMPLE 2

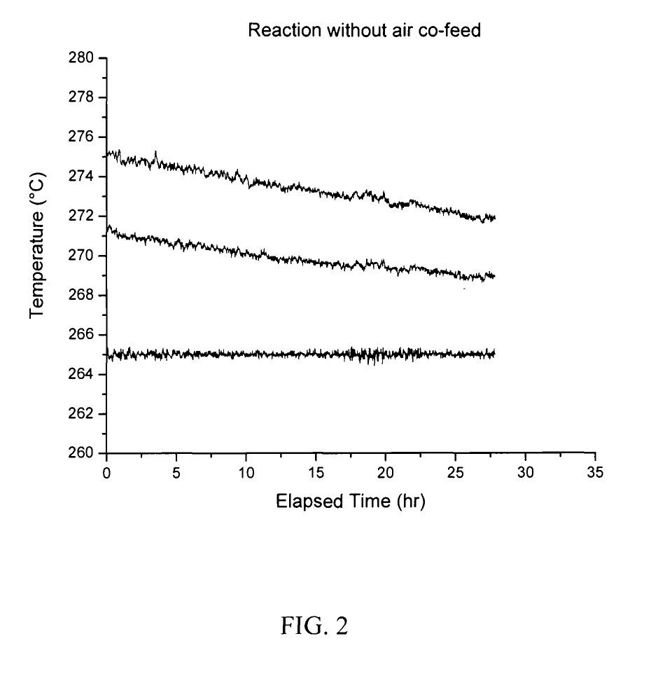

Example 2 is a comparative example intended to illustrate the effect of oxygen co-feed on the chromium oxide catalyst stability during the continuous vapor phase fluorination reaction of 1,1,2,3-tetrachloropropene (HCO-1230xa)+3HF→2-chloro-3,3,3-trifluoropropene (1233xf)+3HCl. For this example the same reaction system and reaction conditions are used as in the Example 1 with the exception that at the completion of the experiment for Example 1, the air co-feed is stopped. After the air co-feed is stopped the temperature of the external heater (bottom line of FIG. 2) is adjusted to bring the catalyst bed temperature, 14 inches from the reactor inlet, to about 270-280° C. Then, as in Example 1, the catalyst bed temperatures 11 and 14 inches from the reactor inlet are monitored for over 20 hours. It is calculated that the temperature at 11 inches (middle line of FIG. 2) is decreasing linearly at the rate of 0.08021° C./hr and the temperature at 14 inches (top line of FIG. 2) is decreasing linearly at the rate of 0.11550° C./hr (FIG. 2).

Temperature measured at 11 and 14 inches inside catalyst bed in the absence of air co-feed decreases 1.6 and 2.3 times faster, respectively, than in the presence of air-co-feed. This indicates that the co-feed of oxygen together with HCO-1230xa and HF to the fluorination reactor, even at a ratio of O₂ to 1,1,2,3-tetrachloropropene (HCO-1230xa) as low as 0.032 to 1 significantly, (at least twofold), decreases the rate of chromium oxide catalyst deactivation.

We claim:

1. A method of preparing fluorinated organic compounds comprising contacting at least one chlorocarbon, selected from the group consisting of 1,1,2,3-tetrachloropropene, 1,1,1,2,3-pentachloropropane and combinations thereof, with a halogenating agent in the presence of at least one catalyst and an oxygen feed under conditions effective to produce a 2-chloro-3,3,3,-trifluoropropene.

2. The method of claim 1 wherein said chlorocarbon is 1,1,2,3-tetrachloropropene or 1,1,1,2,3-pentachloropropane.

3. The method of claim 1 wherein said chlorocarbon is a mixture of 1,1,2,3-tetrachloropropene or 1,1,1,2,3-pentachloropropane.

4. The method of claim 1 wherein said halogenating agent is a fluorinating agent.

5. The method of claim 4 wherein said fluorinating agent comprises hydrogen fluoride.

6. The method of claim 1 wherein the source of oxygen is selected from the group consisting of oxygen gas, dry air, and oxygen gas diluted with an inert gas.

7. The method of claim 1 wherein at least a portion of said contacting step is conducted at a temperature of from about 150° C. to about 450° C.

8. The method of claim 1 wherein at least a portion of said contacting step is conducted at a pressure of from about 0 to about 200 psig.

9. The method of claim 1 wherein the mole ratio of the halogenating agent to the chlorocarbon is greater than or equal to 3:1.

10. The method of claim 9 wherein the mole ratio is between 5:1 and 20:1.

11. The method of claim 1 wherein the mole ratio of the oxygen feed to the chlorocarbon is less than or equal to 0.1:1.

12. The method of claim 11 wherein the mole ratio is between 0.07:1 and 0.005:1.

13. The method of claim 11 wherein the mole ratio is between 0.01:1 and 0.05:1.

14. The method of claim 1 wherein said contacting step comprises conducting at least a portion of said contacting step in the gas phase.

15. The method of claim 1 wherein said catalyst comprises at least one fluorination catalyst.

16. The method of claim 15 wherein the catalyst is selected from the group consisting of chromium, aluminum, cobalt, manganese, nickel and iron oxides, hydroxides, halides, oxyhalides, inorganic salts thereof and their mixtures.

17. The method of claim 15 where the at least one fluorination catalyst is selected from the group consisting of Cr₂O₃, FeCl₃/C, Cr₂O₃, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, NiCl₂/AlF₃, SnCl₄/C, TaCl₅/C, SbCl₃/C, AlCl₃/C, AlF₃/C and combinations thereof.

18. The method of claim 15 wherein at least one fluorination catalyst comprises amorphous Cr₂O₃.

19. The method of claim 1 wherein said catalyst in the presence of said oxygen feed is operable for a greater period of time than said catalyst wherein said oxygen feed is not present.

20. The method of claim 19 wherein said catalyst in the presence of said oxygen feed is substantially operable at least about two fold longer than said catalyst wherein said oxygen feed is not present.
